(19) United States
(12) Patent Application Publication
(10) Pub. No.: US 2011/0269166 A1
(43) Pub. Date: Nov. 3, 2011

(54) SEALING SURFACE SAMPLING PROBE APPLIED TO BLOOD SPOTS AND TISSUES

(75) Inventors: Gary J. Van Berkel, Clinton, TN (US); Vilmos Kertesz, Knoxville, TN (US)

(73) Assignee: UT-BATTELLE, LLC, Oak Ridge, TN (US)

(21) Appl. No.: 12/771,736

(22) Filed: Apr. 30, 2010

(51) Int. Cl. C12Q 1/02; C12N 5/078; C12M 1/34

(52) U.S. Cl. 435/29; 435/287.1; 435/325

(57) ABSTRACT

A sample assembly includes a stack of a sample and a hydrophobic elastic layer located on the back side surface thereof or a filter layer. The sample may include a biological material or a chemical, and can include an absorptive material. The filter layer includes an absorptive material in which a sample material is embedded. A sealing surface sampling probe including a knife edge lands on an area of interest in the sample or the filter layer. The hydrophobic elastic layer or the substrate in conjunction with the knife edge provides a complete sealing of a confined portion of the sample or the filter layer to prevent inclusion of any other material from outside the confined volume and to enhance the precision and sensitivity of the analysis.

### STATEMENT REGARDING FEDERALLY SPONSORED RESEARCH OR DEVELOPMENT

This invention was made with government support under Contract No. DE-AC05-00OR22725 awarded by the U.S. Department of Energy. The government has certain rights in this invention.

### FIELD OF THE INVENTION

The present invention relates to systems including a sample assembly including an hydrophobic elastic layer or a filter layer and configured for operation in combination with a sealing surface sampling probe (SSSP), and methods of operating the same.

### BACKGROUND OF THE INVENTION

Many types of surface sampling probes have been employed to provide a stream of analytes into an analytic instrumentation such as a mass spectrometer. Such surface sampling probes include probes employing thermal desorption, ionization, electrospray, sonic spray, and confined liquid extraction. Probes employing confined liquid extraction generate a stream of eluate, which includes the material from the sample subjected to surface sampling within the liquid initially directed at a surface of the sample. These probes are collectively referred to as liquid extraction surface sampling probes. Examples of liquid extraction surface sampling probes include sealing surface sampling probes (SSSP's) and liquid microjunction surface sampling probes (LMJ-SSP's). Sealing surface sampling probes extract the eluate by confining a liquid stream with a sealed surface contact. Liquid microjunction surface sampling probes extract the eluate using a confined liquid stream with liquid microjunction surface contact.

Details of sealing surface sampling probes are provided in International Patent Application Publication WO 02/08747 A1 to Heinrich Luftmann, Heinrich Luftmann, Mario Aranda, and Gertrud E. Morlock, “Automated Interface for Hyphenation of Planar Chromatography with Mass Spectrometry,” Rapid Commun. Mass Speetrom., Vol. 21, Issue 23, pp. 3772-3776 (2007) and Heinrich Luftmann, “A Simple Device for the Extraction of TLC Spots: Direct Coupling with an Electrospray Mass Spectrometer,” Anal. Bioanal. Chem. Vol. 378, No. 4, pp. 964-968 (2004).

Prior art methods employ a sample on a hard substrate, which is typically a glass slide, for analysis with a sealing surface sampling probe. At a microscopic level, the hard surface is not uniform, and any contact between a protruding portion of the sealing surface sampling probe and the contacting surface of the hard substrate necessarily forms microscopic lateral openings around the area of the sample to be analyzed. The imperfections in the “sealing” during the operation of the sealing surface sampling probe results in lateral extrusion and diffusion of the eluting solvent, especially when the sample includes an absorptive material. Such lateral extrusion and diffusion of the eluting solvent results in smearing of the sample, deterioration of the spatial resolution of the analysis, inclusion of reverse-diffused material from surrounding areas, and/or reduction of sensitivity of the analysis.

### SUMMARY OF THE INVENTION

A sample assembly includes a stack of a sample and a hydrophobic elastic layer located on the back side surface thereof or a filter layer. The sample may include a biological material or a chemical, and can include an absorptive material. The filter layer includes an absorptive material in which a sample material is embedded. A sealing surface sampling probe including a knife edge lands on an area of interest in the sample or the filter layer.

In one embodiment, the knife edge cuts through a periphery of the area of interest into an upper portion of the hydrophobic elastic layer to form a confined portion of the sample, which is laterally confined by the knife edge and vertically confined by the sealing surface sampling probe and the upper surface of the hydrophobic elastic layer. In another embodiment, the knife edge cuts through a periphery of the area of interest into an upper portion of the substrate to form a confined portion of the filter layer, which is laterally confined by the knife edge and vertically confined by the sealing surface sampling probe and the upper surface of the hydrophobic elastic layer. The hydrophobic elastic layer or the substrate in conjunction with the knife edge provides a complete sealing of the confined portion of the sample or the filter layer to prevent inclusion of any other material from outside the confined volume and to enhance the precision and sensitivity of the analysis.

According to an aspect of the present invention, a system for generating a stream of eluate from a sample is provided. The system includes: a sample assembly including a sample and a hydrophobic elastic layer contacting a back side surface of the sample; and a sealing surface sampling probe configured to feed a liquid to a surface of a portion of the sample, wherein the sealing surface sampling probe includes a knife edge configured to cut into a periphery of the portion of the sample to contact the hydrophobic elastic layer, wherein the portion of the sample is encapsulated by the hydrophobic elastic layer, the knife edge, and the liquid.

In one embodiment, the hydrophobic elastic layer consists essentially of at least one hydrophobic elastomer, at least one hydrophobic resin, or a combination thereof.

In another embodiment, the hydrophobic elastic layer is a hydrophobic elastic tape having a substantially uniform thickness.

In even another embodiment, the hydrophobic elastic layer has a thickness from 300 micron to 6 mm.

In yet another embodiment, the sample assembly further includes a pad that provides mechanical support under external pressure of at least 200 psi, and the hydrophobic elastic layer is a hydrophobic coating located directly on a surface of the pad.

In still another embodiment, the hydrophobic coating has a thickness from 3 microns to 300 microns.

In a further embodiment, the sample assembly further includes a substrate located on a back side of the hydrophobic elastic layer and providing mechanical support to the hydrophobic elastic layer.

In an even further embodiment, the portion of the sample is laterally enclosed by the knife edge and has a cross-sectional shape of a circle, an ellipse, a superellipse, or a polygonal shape.

In a yet further embodiment, the portion of the sample is laterally enclosed by the knife edge and has an area from 2 mm² to 80 mm².

In a still further embodiment, the knife edge includes a material selected from a metal or a metallic alloy.

In further another embodiment, the sealing surface sampling probe includes at lease one inlet for letting in the liquid and an outlet for letting out the stream of eluate.

In even further another embodiment, the sample is a slice of a biological tissue.

In yet further another embodiment, the biological tissue has a thickness from 4 microns to 400 microns.

In still further another embodiment, the liquid is a solvent that dissolves the portion of the sample.

According to another aspect of the present invention, a method of generating a stream of eluate from a sample is provided. The method includes: forming a sample assembly including a sample and a hydrophobic elastic layer contacting a back side surface of the sample; bringing a sealing surface sampling probe into contact with the sample assembly, wherein a knife edge cuts into a periphery of a portion of the sample to contact the hydrophobic elastic layer; and generating a stream of eluate from the portion of the sample by feeding a liquid to the portion of the sample, wherein the portion of the sample is encapsulated by the hydrophobic elastic layer, the knife edge, and the liquid, and wherein the stream of eluate includes the liquid and materials of the portion of the sample.

In one embodiment, the hydrophobic elastic layer consists essentially of at least one hydrophobic elastomer, at least one hydrophobic resin, or a combination thereof.

In another embodiment, the hydrophobic elastic layer is a hydrophobic elastic tape having a substantially uniform thickness.

In even another embodiment, the hydrophobic elastic layer has a thickness from 300 micron to 6 mm.

In yet another embodiment, the sample assembly further includes a pad that provides mechanical support under external pressure of at least 200 psi, and the hydrophobic elastic layer is a hydrophobic coating located directly on a surface of the pad.

In still another embodiment, the hydrophobic coating has a thickness from 3 microns to 300 microns.

In a further embodiment, the sample assembly further includes a substrate located on a back side of the hydrophobic elastic layer and providing mechanical support to the hydrophobic elastic layer.

In an even further embodiment, the portion of the sample is laterally enclosed by the knife edge and has a cross-sectional shape of a circle, an ellipse, a superellipse, or a polygonal shape.

In a yet further embodiment, the portion of the sample is laterally enclosed by the knife edge and has an area from 2 mm² to 80 mm².

In a still further embodiment, the knife edge includes a material selected from a metal or a metallic alloy.

In further another embodiment, the sealing surface sampling probe includes at lease one inlet for letting in the liquid and an outlet for letting out the stream of eluate.

In even further another embodiment, the sample is a slice of a biological tissue.

In yet further another embodiment, the biological tissue has a thickness from 4 microns to 400 microns.

In still further another embodiment, the liquid is a solvent that dissolves the portion of the sample.

According to yet another aspect of the present invention, a system for generating a stream of eluate is provided. The system includes: a sample assembly comprising a filter layer; and a sealing surface sampling probe configured to feed a liquid to a surface of a portion of the filter layer, wherein the sealing surface sampling probe includes a knife edge configured to cut into a periphery of the portion of the filter layer, wherein the portion of the filter layer is laterally confined by the knife edge.

In one embodiment, the sample assembly may further include a substrate contacting a back side surface of the filter layer. The substrate may have a hydrophobic surface that contacts the back side surface of the filter layer.

In another embodiment, the portion of the filter layer is laterally enclosed by the knife edge and has a cross-sectional shape of a circle, an ellipse, a superellipse, or a polygonal shape.

In yet another embodiment, the knife edge includes a material selected from a metal or a metallic alloy.

In still another embodiment, the sample is a blood spot paper.

In a further embodiment, the liquid is a solvent that dissolves blood.

According to still another aspect of the present invention, a method of generating a stream of eluate is provided. The method includes: forming a sample assembly comprising a filter layer; bringing a sealing surface sampling probe into contact with the sample assembly, wherein a knife edge cuts into a periphery of a portion of the filter layer; and generating a stream of eluate from the portion of the filter layer by feeding a liquid to the portion of the filter layer, wherein the portion of the filter layer is laterally confined by the knife edge, and wherein the stream of eluate includes the liquid and materials of the portion of the filter layer.

In one embodiment, the sample assembly may further include a substrate contacting a back side surface of the filter layer. The substrate may have a hydrophobic surface that contacts the back side surface of the filter layer.

In another embodiment, the portion of the filter layer is laterally enclosed by the knife edge and has a cross-sectional shape of a circle, an ellipse, a superellipse, or a polygonal shape.

In yet another embodiment, the knife edge includes a material selected from a metal or a metallic alloy.

In still another embodiment, the sample is a blood spot paper.

In a further embodiment, the liquid is a solvent that dissolves blood.

According to a further aspect of the present invention, a system for analyzing a composition of an eluate is provided. The system includes: a sample assembly comprising a filter layer or a combination of a sample and a hydrophobic elastic layer contacting a back side surface of the sample; a sealing surface sampling probe configured to generate a stream of eluate from the sample assembly, wherein the sealing surface sampling probe includes a knife edge configured to laterally confine a portion of the sample or the filter layer, wherein the portion is laterally confined by the knife edge; and an analytical instrument configured to receive and analyze the stream of eluate.

In one embodiment, the system further includes an ionization source configured to receive the stream of eluate from the sealing surface sampling probe and to provide an ionized material of the eluate to the analytical instrument.

According to a yet further aspect of the present invention, a method of analyzing a composition of an eluate is provided. The method includes: forming a sample assembly comprising a filter layer or a combination of a sample and a hydrophobic elastic layer contacting a back side surface of the sample; bringing a sealing surface sampling probe into contact with the sample assembly, wherein a knife edge cuts into a periphery of a portion of the sample assembly; generating a stream of eluate from the portion by feeding a liquid to the portion, wherein the portion is laterally confined by the knife edge, and wherein the stream of eluate includes the liquid and materials of the portion; and analyzing the steam of eluate employing an analytical instrument.

In one embodiment, the method further includes: generating an ionized material from the stream of eluate; and providing the ionized material to the analytical instrument.

### BRIEF DESCRIPTION OF THE DRAWINGS

FIG. 1 is a cross-sectional view of a sample assembly according to a first embodiment of the present invention.

FIGS. 2A-2E are sequential schematic views illustrating the operation of a sealing surface sampling probe on the sample assembly according to the first embodiment of the present invention. Arrows show the direction of the liquid flow

FIGS. 3A-3E are sequential magnified views of the sample and the hydrophobic elastic layer at processing steps corresponding to FIGS. 2A-2E, respectively, according to the first embodiment of the present invention.

FIG. 4 is a cross-sectional view of a sample assembly according to a second embodiment of the present invention.

FIGS. 5A-5E are sequential schematic views illustrating the operation of a sealing surface sampling probe on the sample assembly according to the second embodiment of the present invention. Arrows show the direction of the liquid flow

FIGS. 6A-6E are sequential magnified views of the sample and the hydrophobic elastic layer at processing steps corresponding to FIGS. 5A-5E, respectively, according to the second embodiment of the present invention.

FIG. 7A is a selected reaction monitoring (SRM) ion current chronograms of sitamaquine (m/z 344.4→271.2, CE=30 eV) obtained from the analysis of dried rat blood spot calibration standards using positive ion mode electrospray ionization (ESI).

FIG. 7B is a selected reaction monitoring (SRM) ion current chronograms of sitamaquine-d10 (m/z 354.4→271.2, CE=30 eV) obtained from the analysis of dried rat blood spot calibration standards using positive ion mode ESI as in FIG. 7A.

FIG. 7C is a calibration curve constructed using calibration standards (line) and average of QC samples (O) with error bars (CV) using the ratio of background corrected integrated (over the 60 s sampling period) SRM signal of sitamaquine (10-10000 ng/mL) and that of sitamaquine-d10 (570 ng/mL) (A_{sitamaqine}/A_{sitamaquine-d10}) as a function of sitamaquine concentration (c_{sitamaquine}) in the blood spotted onto the paper substrate based on the data of FIGS. 7A and 7B.

FIG. 8A is a selected reaction monitoring (SRM) ion current chronograms of acetaminophen (m/z 152.1→110.1, CE=40 eV) obtained from the analysis of dried human blood spot calibration standards using positive ion mode atmospheric pressure chemical ionization (APCI).

FIG. 8B is a selected reaction monitoring (SRM) ion current chronograms of acetaminophen-d4 (m/z 156.1→114.1, CE=40 eV) obtained from the analysis of dried human blood spot calibration standards using positive ion mode APCI as in FIG. 8A.

FIG. 8C is a calibration curve constructed using calibration standards (line) and average of QC samples (O) with error bars (CV) using the ratio of background corrected integrated (over the 1 min sampling period) SRM signal of acetaminophen (50-5000 ng/mL) and that of acetaminophen-d4 (7000 ng/mL) (A_{acetaminophen}/A_{acetamophen-d4}) as a function of acetaminophen concentration (c_{acetaminophen}) in the blood spotted onto the paper substrate based on the data of FIGS. 8A and 8B.

FIG. 9A is photograph of a propranolol dosed mouse (7.5 mg/kg, I.V. dosed, sacrificed 1 hour after dose) whole-body thin tissue section on adhesive tape. The six discrete points analyzed are annotated: 1=liver; 2=brain; 3=kidney; 4=stomach/contents; 5=heart; 6=lung.

FIG. 9B is an SRM ion current chronograms for propranolol (m/z 260→183, CE=27 eV) recorded during a 60 s sampling period at each point using positive ion mode ESI.

FIG. 9C is an SRM ion current chronograms for hydroxypropranolol glucuronide (m/z 452→276, CE=35 eV) recorded during a 60 s sampling period at each point using positive ion mode ESI.

FIG. 9D shows average integrated area with error bars (CV) of the SRM signals of propranolol for organs analyzed in two separate tissue sections of the whole-body thin tissue section of FIG. 9A. Dwell time was 50 ms for each transition monitored. Extraction solvent was 80/20/0.1 (v/v/v) acetonitrile/water/formic acid at a flow rate of 200 μL/min.

FIG. 9E shows average integrated area with error bars (CV) of the SRM signals of hydroxypropranolol glucuronide for organs analyzed in two separate tissue sections of the whole-body thin tissue section of FIG. 9A.

### DETAILED DESCRIPTION OF THE INVENTION

As stated above, the present invention relates to systems including a sample assembly including an hydrophobic elastic layer or a filter layer and configured for operation in combination with a sealing surface sampling probe (SSSP), and methods of operating the same, which are now described in detail with accompanying figures. It is noted that like and corresponding elements mentioned herein and illustrated in the drawings are referred to by like reference numerals. It is also noted that proportions of various elements in the accompanying figures are not drawn to scale to enable clear illustration of elements having smaller dimensions relative to other elements having larger dimensions.

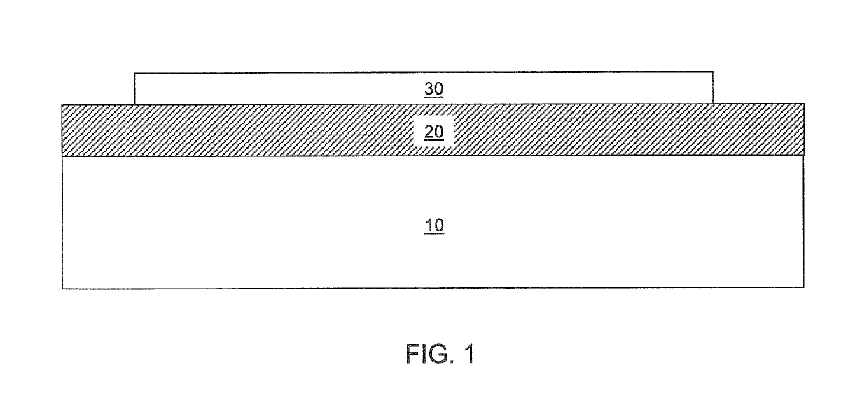

Referring to FIG. 1, a sample assembly according to a first embodiment of the present invention includes stack of a sample 30 and a hydrophobic elastic layer 20. The sample 30 may include a porous material, a non-porous material, or a combination thereof. Further, the sample 30 may include an absorptive material, a non-absorptive material, or a combination thereof. The sample 30 may include a biological material or a chemical. For example, the sample may include a piece of paper, a piece of cloth, a porous ceramic material, a biological tissue, a high performance thin layer chromatography (HPTLC) plate used in chromatography, a blood spot paper, or any other structure including a material that can be dissolved in a liquid, i.e., a solvent.

Biological materials that can be embedded in the sample region 30 include human blood, animal blood, serum, and any other liquid excreted or extracted from a biological cell. The thickness of the sample 30 can be from 10 micron to 1 cm, and typically from 50 micron to 1 mm, although lesser and greater thicknesses can also be employed. The sample 30 can be a slice of a biological tissue. For example, the biological tissue can have a thickness from 4 microns to 400 microns.

The hydrophobic elastic layer 20 includes an elastic material and has a hydrophobic surface that contacts a planar back side surface of the sample 30. The hydrophobic elastic layer 20 includes at least one hydrophobic elastomer, at least one hydrophobic resin, or a combination thereof. In one embodiment, the hydrophobic elastic layer 20 consists essentially of at least one hydrophobic elastomer, at least one hydrophobic resin, or a combination thereof.

The hydrophobic elastic layer 20 can be a hydrophobic elastic tape having a substantially uniform thickness. For example, the hydrophobic elastic layer 20 can have a thickness from 300 micron to 6 mm. An example of the hydrophobic elastic layer 20 is a commercially available acetate tape such as the Scotch® Magic™ No. 810 tape (commonly referred to as the “Scotch tape”) by 3M™.

In one embodiment, the sample assembly can further include a pad (not shown) that provides mechanical support under external pressure of at least 200 psi. In this case, the hydrophobic elastic layer 20 can be a hydrophobic coating located directly on a surface of the pad. For example, the hydrophobic coating can have a thickness from 3 microns to 300 microns.

The sample assembly may, or may not, further include a substrate 10 located on a back side of the hydrophobic elastic layer 20 or the pad. In other words, the presence of the substrate 10 is optional. The substrate 10 provides mechanical support to the hydrophobic elastic layer 20 during an extraction operation employing a liquid extraction surface sampling probe. The substrate 10 includes a rigid material such as a metallic material or a material having an equivalent Young's modulus.

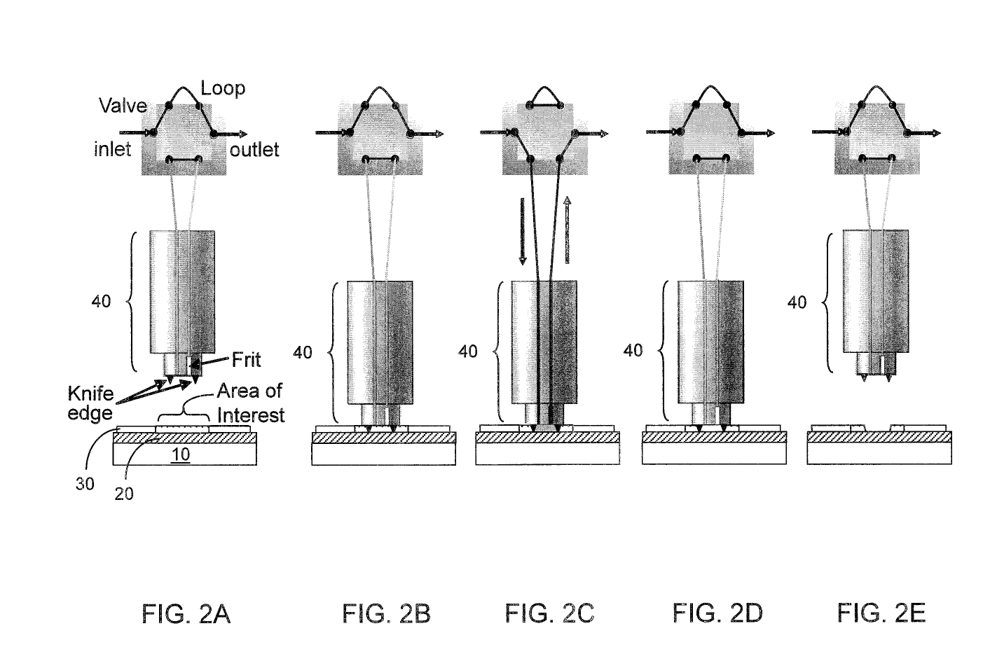

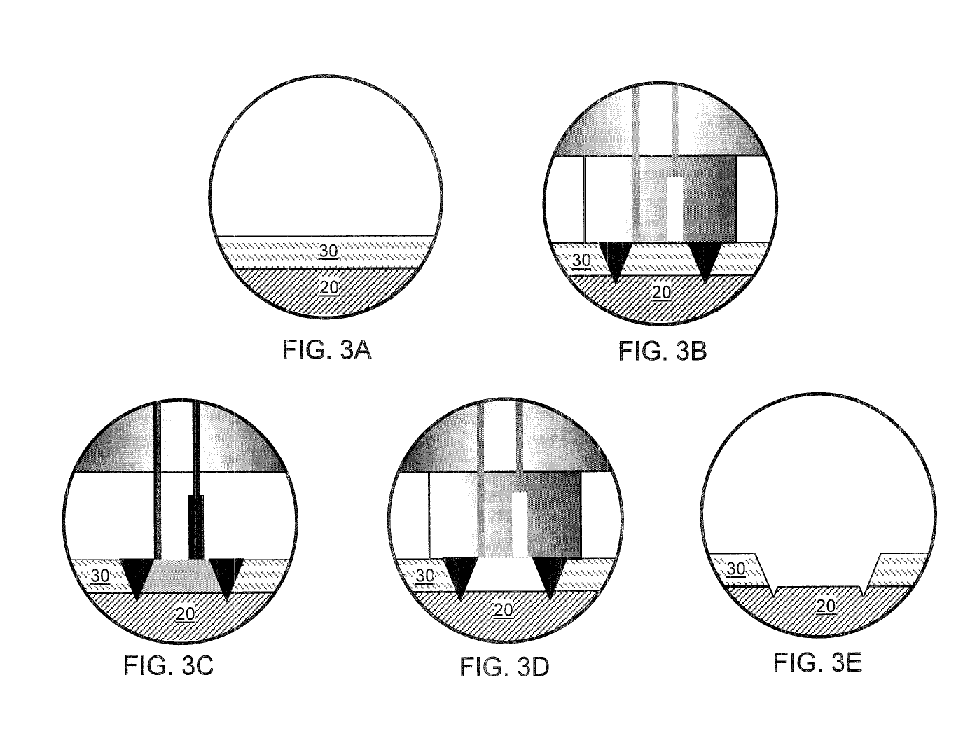

FIGS. 2A-2E are sequential diagrams illustrating the operation of a system including a liquid extraction surface sampling probe 40 and the sample assembly (10, 20, 30). Arrows show the direction of the flow of the liquid, which can be a solvent for dissolving the material of the sample 30. FIGS. 3A-3E are magnified views of an area of interest within the sample 30 and regions in the immediate vicinity at processing steps corresponding to FIGS. 2A-2E, respectively.

Referring to FIGS. 2A and 3A, the sample assembly (10, 20, 30) is brought to a position under the liquid extraction surface sampling probe 40 such that the area of interest within the sample 30 directly underlies the head, or the “plunger,” of the liquid extraction surface sampling probe 40. The liquid extraction surface sampling probe 40 can be a sealing surface sampling probe including a knife edge. The sealing surface sampling probe can be configured to feed a liquid to a surface of a portion of the sample, which is typically the area of interest of the sample 30.

The knife edge is located on a proximal surface of the liquid extraction surface sampling probe 40, i.e., on the surface of the face that is brought in proximity to the sample assembly (10, 20, 30). The knife edge defines a contiguous closed shape, which can be a circle, an ellipse, a superellipse, or a polygon. The closed shape of the knife edge is the shape of a sample area to be subsequently analyzed. The knife edge is composed of an impermeable material such as a metal, a metallic compound, or any other impermeable dielectric or conductive material that does not let liquids leak through.

The liquid extraction surface sampling probe 40 includes at lease one inlet for letting in the liquid and an outlet for letting out the stream of eluate. Prior to analysis of the sample 10, the liquid extraction surface sampling probe 40 is configured to cause the liquid supplied to the inlet of the liquid extraction surface sampling probe 40 to bypass the head of the liquid extraction surface sampling probe 40. The bypassing of the head can be effected by a bypass loop between the inlet and the outlet of the liquid extraction surface sampling probe 40. The liquid flow through the bypass loop can be effected by actuation of at least one flow control valve.

Referring to FIGS. 2B and 3B, the liquid extraction surface sampling probe 40 is brought into contact with the sample 30 at the area of interest. The knife edge is configured to cut into a periphery of the portion of the sample and to contact the hydrophobic elastic layer 20. The knife edge punctures through the sample 30 to protrude into the hydrophobic elastic layer 20, thereby forming an enclosed volume vertically confined between the proximal face of the head of the liquid extraction surface sampling probe 40 and the top surface of the hydrophobic elastic layer 20 and laterally confined at a periphery defined by the inner surface of the knife edge. The contact between the knife edge of the liquid extraction surface sampling probe 40 and the hydrophobic elastic layer 20 can be effected by an upward vertical movement of the sample assembly (10, 20, 30), a downward vertical movement of the head of the liquid extraction surface sampling probe 40, or a combination thereof. The vertical dimension of the knife edge is greater than the thickness of the sample 30 to ascertain that the knife edge cuts into the top surface of the hydrophobic elastic layer 20. The hydrophobic property of the top surface of the hydrophobic elastic layer 20 in combination with the impermeability of the knife edge and the sealing geometry provided by the head of the liquid extraction surface sampling probe 40 prevents loss of the sample material within the confined volume by leakage during the subsequent analysis step.

Referring to FIGS. 2C and 3C, the liquid is fed to the confined portion of the sample 30 by changing the valve configuration at the inlet and/or at the outlet. The liquid can be fed to the confined portion of the sample 30, for example, through an annular capillary and directed to the outlet through an inner capillary. Alternately, any other capillary configuration can be employed provided that at least one capillary directs the flow of the liquid from the inlet to the confined portion of the sample 30 and at least another capillary directs the flow of the liquid from the confined portion of the sample 30 to the outlet. The confined portion of the sample 30 is encapsulated by the hydrophobic elastic layer 20, the knife edge, and the liquid during the extraction step. The confined portion of the sample 30 is laterally enclosed by the knife edge, and has a cross-sectional shape that is identical to the inside of the horizontal cross-sectional shape of the knife edge. For example, the confined portion of the sample 30 can have a cross-sectional shape of a circle, an ellipse, a superellipse, or a polygonal shape. The confined portion of the sample 30 can have an area from 2 mm² to 80 mm².

The liquid from the inlet to the sample 30 does not include any dissolved material from the sample 30, and is herein referred to as an eluent or an eluting solvent. The liquid in the sample region 30 dissolves the embedded material of the sample 30. The liquid from the sample 30 to the outlet includes dissolved materials from the sample 30, and is herein referred to as an eluent. Typically, the liquid is a solvent that is capable of dissolving the embedded sample material in the sample 30. The liquid that comes out of the outlet forms a stream of eluate. The composition of the eluate includes the liquid of the eluent and the dissolved material that originates from the material of the confined volume of the sample 30. The stream of eluate contiguously flows out of the outlet of the liquid extraction surface sampling probe 40. The outlet can be located within a device for ionizing the liquid. Further, the device for ionizing the liquid can be selected from an electrospray ionization device, an atmospheric chemical ionization device, an inductively coupled plasma ionization device, and an atmospheric photoionization device.

Referring to FIGS. 2D and 3D, continuous operation of the liquid extraction surface sampling probe 40 eventually removes all materials within the confined volume of the sample 30. At this point, the valve(s) is/are actuated again to change the liquid flow path, i.e., to bypass the head of the liquid extraction surface sampling probe 40 and to cause the liquid to flow through the bypass loop.

Referring FIGS. 2E and 3E, the head of the liquid extraction surface sampling probe 40 is vertically separated from the remaining portion of the sample 30. The vertical separation can be effected by a vertical movement of the head of the liquid extraction surface sampling probe 40, a vertical movement of the sample assembly (10, 20, 30), or a combination thereof. Typically, the top surface of the hydrophobic elastic layer 20 has indentations that correspond to the protrusion of the knife edge therein during the extraction run.

The stream of eluate that contiguously flows out of the outlet of the liquid extraction surface sampling probe 40 at the step of FIGS. 2C and 3C can be routed to any analytical instrument capable of determining the composition of the material in the stream of eluate. For example, the analytical instrument can be a mass spectrometer that determines the m/z ratio of the components of the stream of eluate. The stream of eluate can be supplied to the analytical instrument continuously in real time, continuously with a predetermined or programmable time delay, or intermittently through a temporary storage unit.

In case the stream of eluate is ionized for analysis, various ionization techniques may be employed. Further, additional treatment, preparation, or handling may be performed between the extraction from the surface and the ionization of the analytes that is in the eluant. In case a mass spectrometer is employed, the eluant flow can be directed into an ionization region of a mass spectrometer without modification, or can be directed into the mass spectrometer after modified by any known methods, such as preconcentration and cleanup on a suitable stationary phase or HPLC separation of the analytes extracted. Ionization sources that can be utilized include all liquid introduction ionization sources such as an electrospray ionization (ESI) device, an atmospheric pressure chemical ionization (APCI) device, an atmospheric pressure photo ionization (APPI) device, and an inductively coupled plasma ionization (ICPI) device. The ionization source is configured to receive the stream of eluate from the sealing surface sampling probe and to generate and to provide an ionized material of the eluate to the analytical instrument.

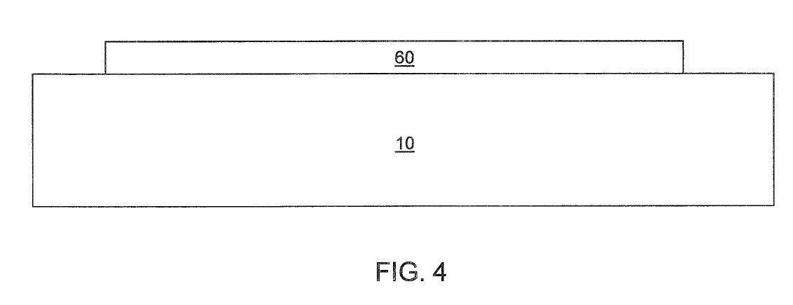

Referring to FIG. 4, a sample assembly according to a second embodiment of the present invention includes stack of a filter layer 60. The filter layer 60 includes an absorptive material. The absorptive material in the filter layer 60 may be a porous material. For example, the filter layer 60 may include a piece of paper, a piece of cloth, or a porous ceramic material, a blood spot paper, or any other absorptive structure including a material that can be dissolved in a liquid, i.e., a solvent. The sample assembly may, or may not, further include a substrate 10 located on a back side of the filter layer 60. Thus, the presence of the substrate 10 is optional.

In one embodiment, the filter layer 60 includes a sample region in which a foreign material is embedded. The embedded foreign material can be any material that can be absorbed into the filter layer 60. Non-limiting examples of the embedded foreign material include human blood, animal blood, serum, and any other liquid excreted or extracted from a biological organism or cell.

The filter layer 60 can include a filter paper. In case the filter layer 60 includes a filter paper, the filter paper can be a blood spot paper including wet or dry blood or any other liquid excreted or extracted from a biological organism or cell. The filter paper may be of the types that are widely used for large screening programs in medical facilities or biology laboratories. The filter paper may be a cellulose-based filter paper suitable for blood sampling.

If the sample assembly further includes a substrate 10 on a back side of the filter layer 60, the substrate 10 provides mechanical support to the filter layer 60 during an extraction operation employing a liquid extraction surface sampling probe. The substrate 10 includes a rigid material such as a metallic material or a material having an equivalent Young's modulus.

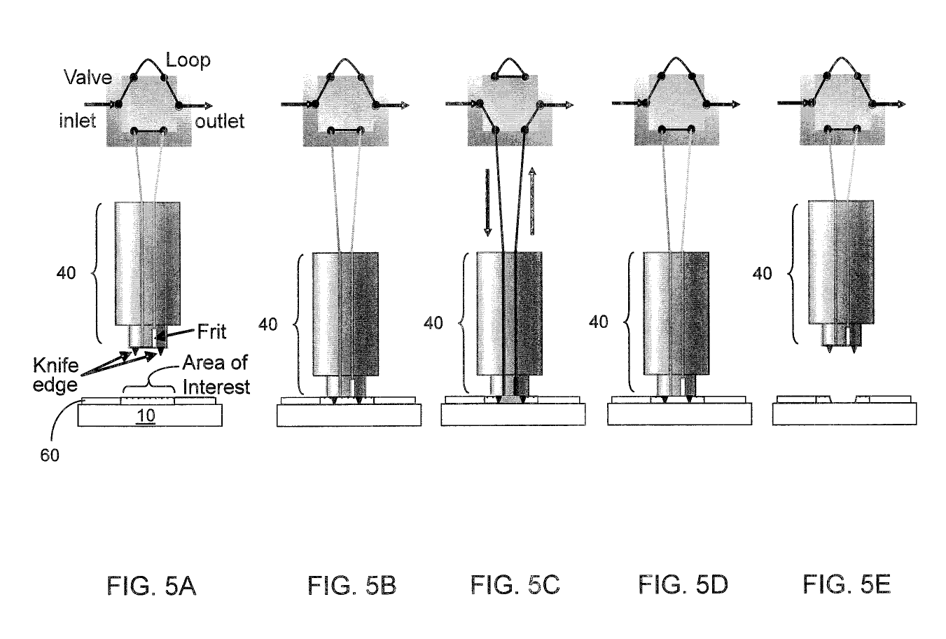

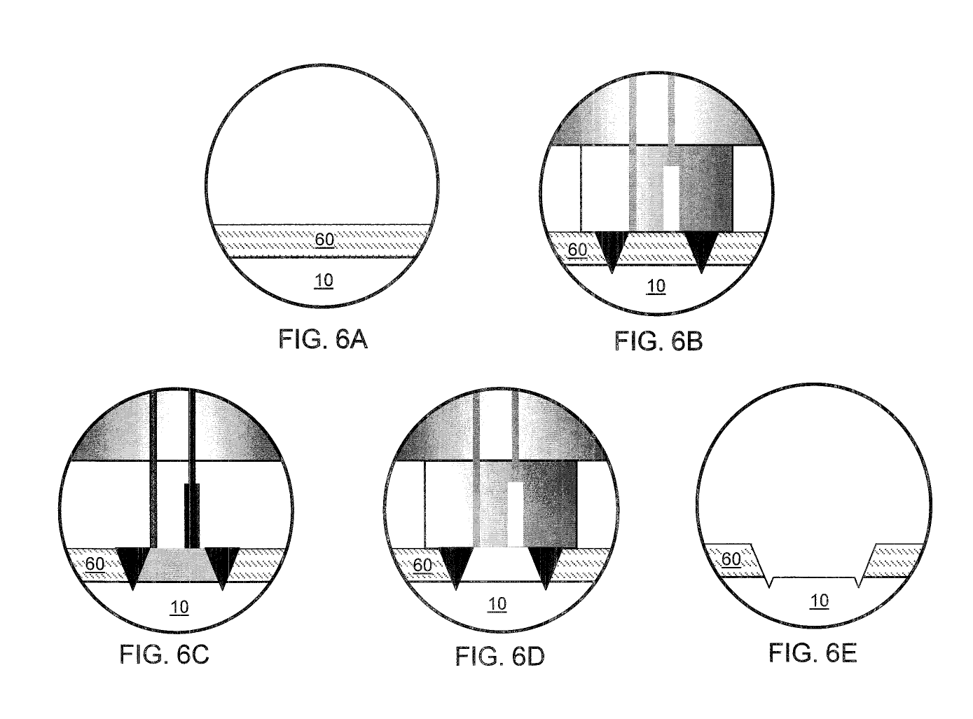

FIGS. 5A-5E are sequential diagrams illustrating the operation of a system including a liquid extraction surface sampling probe 40 and the sample assembly (10, 60). Arrows show the direction of the flow of the liquid, which can be a solvent for dissolving the embedded material in the filter layer 60. FIGS. 6A-6E are magnified views of an area of interest within the filter layer 60 and regions in the immediate vicinity at processing steps corresponding to FIGS. 5A-5E, respectively.

Referring to FIGS. 5A and 6A, the sample assembly (10, 60) is brought to a position under the liquid extraction surface sampling probe 40 such that the area of interest within the filter layer 60 directly underlies the head of the liquid extraction surface sampling probe 40. For example, if the filter layer 60 is a blood spot paper, the area of interest can be the area in which blood or any other sample material is embedded. The liquid extraction surface sampling probe 40 can be a sealing surface sampling probe including a knife edge. The sealing surface sampling probe can be configured to feed a liquid to a surface of a portion of the sample, which is typically the area of interest of the filter layer 60. The sealing surface sampling probe may have the same construction and/or configuration as in the first embodiment.

Referring to FIGS. 5B and 6B, the liquid extraction surface sampling probe 40 is brought into contact with the filter layer 60 at the area of interest. The knife edge is configured to cut into a periphery of the portion of the filter layer 60 and to contact the substrate 10. The knife edge punctures through the filter layer 60 to protrude into an upper portion of the substrate 10, thereby forming an enclosed volume vertically confined between the proximal face of the head of the liquid extraction surface sampling probe 40 and the top surface of the substrate 10 and laterally confined at a periphery defined by the inner surface of the knife edge. The contact between the knife edge of the liquid extraction surface sampling probe 40 and the substrate 10 can be effected by an upward vertical movement of the sample assembly (10, 60), a downward vertical movement of the head of the liquid extraction surface sampling probe 40, or a combination thereof. The vertical dimension of the knife edge is greater than the thickness of the filter layer 60 to ascertain that the knife edge cuts into the top surface of the substrate 10. Optionally but not necessarily, the top surface of the substrate 10 may have hydrophobic property. The sample material subjected to analysis is contained within the confined volume.

Referring to FIGS. 5C and 6C, the liquid is fed to the confined portion of the filter layer 60 by changing the valve configuration at the inlet and/or at the outlet. The liquid can be fed to the confined portion of the filter layer 60, for example, through an annular capillary and directed to the outlet through an inner capillary. Alternately, any other capillary configuration can be employed provided that at least one capillary directs the flow of the liquid from the inlet to the confined portion of the filter layer 60 and at least another capillary directs the flow of the liquid from the confined portion of the filter layer 60 to the outlet. If the substrate 10 is present, the confined portion of the filter layer 60 is encapsulated by the substrate 10, the knife edge, and the liquid during the extraction step. The confined portion of the filter layer 60 is laterally enclosed by the knife edge, and has a cross-sectional shape that is identical to the inside of the horizontal cross-sectional shape of the knife edge. For example, the confined portion of the filter layer 60 can have a cross-sectional shape of a circle, an ellipse, a superellipse, or a polygonal shape. The confined portion of the filter layer 60 can have an area from 2 mm² to 80 mm².

The liquid from the inlet to the sample 30 does not include any dissolved material from the confined portion of the filter layer 60, and constitutes the eluent or the eluting solvent. The liquid in the confined portion of the filter layer 60 dissolves the embedded material within the filter layer 60. The liquid from the confined portion of the filter layer 60 to the outlet includes dissolved materials from the confined portion of the filter layer 60, and constitutes the eluent. Typically, the liquid is a solvent that is capable of dissolving the embedded sample material in the confined portion of the filter layer 60. The liquid that comes out of the outlet forms a stream of eluate. The composition of the eluate includes the liquid of the eluent and the dissolved material that originates from the material of the confined portion of the filter layer 60. The stream of eluate contiguously flows out of the outlet of the liquid extraction surface sampling probe 40. The outlet can be located within a device for ionizing the liquid. Further, the device for ionizing the liquid can be selected from an electrospray ionization device, an atmospheric chemical ionization device, an inductively coupled plasma ionization device, and an atmospheric photoionization device.

Referring to FIGS. 5D and 6D, continuous operation of the liquid extraction surface sampling probe 40 eventually removes all materials within the confined portion of the filter layer 60. At this point, the valve(s) is/are actuated again to change the liquid flow path, i.e., to bypass the head of the liquid extraction surface sampling probe 40 and to cause the liquid to flow through the bypass loop.

Referring FIGS. 5E and 6E, the head of the liquid extraction surface sampling probe 40 is vertically separated from the filter layer 60. The vertical separation can be effected by a vertical movement of the head of the liquid extraction surface sampling probe 40, a vertical movement of the sample assembly (10, 60), or a combination thereof. Typically, the top surface of the substrate 10 has indentations that correspond to the protrusion of the knife edge therein during the extraction run.

The stream of eluate that contiguously flows out of the outlet of the liquid extraction surface sampling probe 40 at the step of FIGS. 5C and 6C can be routed to any analytical instrument capable of determining the composition of the material in the stream of eluate as in the first embodiment. Further, in case the stream of eluate is ionized for analysis, various ionization techniques may be employed as in the first embodiment. Additional treatment, preparation, or handling may be performed between the extraction from the surface and the ionization of the analytes that is in the eluant. In case a mass spectrometer is employed, the eluant flow can be directed into an ionization region of a mass spectrometer without modification, or can be directed into the mass spectrometer after modified by any known methods. The ionization source is configured to receive the stream of eluate from the sealing surface sampling probe and to generate and to provide an ionized material of the eluate to the analytical instrument.

### EXAMPLES

Chemicals:

HPLC grade acetonitrile, methanol and water were purchased from Burdick & Jackson (Muskegon, Mich., USA). Formic acid (>96% purity) was purchased from Sigma-Aldrich (St. Louis, Mo., USA). Propranolol hydrochloride was obtained from Acros Organics (Morris Plains, N.J., USA). Sitamaquine [N,N-diethyl-N-(6-methoxy-4-methylquinolin-8-yl)hexane-1,6-diamine]dihydrochloride and acetaminophen were obtained from GlaxoSmithKline (Greenford, UK) and Sigma-Aldrich, respectively. Isotopically labelled internal standards (ISs) sitamaquine-d10 dihydrochloride and acetaminophen-d4 were produced by Isotope Chemistry, GlaxoSmithKline (Stevenage, UK).

Dried Blood Spot Sample Preparation:

Stock solutions (1 mg/mL) of sitamaquine and sitamaquine-d10 were prepared in 50/50 (v/v) methanol/water. Rat blood was mixed with the analyte stock solutions prior to spotting in order to obtain the required sitamaquine (0-10000 ng/mL) and sitamaquine-d10 (570 ng/mL) concentrations. Stock solutions (1 mg/mL) of acetaminophen and acetaminophen-d4 were prepared in dimethyl formamide. Human blood was mixed with the analyte stock solutions prior to spotting to obtain the required acetaminophen (0-5000 ng/mL) and acetaminophen-d4 (7000 ng/mL) concentrations. Aliquots (15 μL) of the dosed rat (sitamaquine) or human (acetaminophen) blood samples were spotted onto Ahlstrom 237 filter paper (Ahlstrom Corp., Helsinki, Finland) for both the calibration standards and quality control (QC) samples, and allowed to dry at room temperature for at least 2 h. One replicate of each member of a calibration standard set, which included a blank (no analyte, only IS) and total blank (no analyte, no IS) samples, was analyzed before and after of six replicates of each member of the QC samples. Both the standards and. QC samples were analyzed in order of decreasing analyte concentration.

Thin Tissue Section Preparation;

Male CD-1 mice (Charles River Laboratories) were administered propranolol intravenously via the tail vein at 7.5 mg/kg as an aqueous solution in 0.9% NaCl. At 60 min post-dose, mice were euthanized with an isoflurane overdose and immediately frozen in dry ice/hexane. The frozen mice were embedded/blocked in 2% aqueous carboxymethyl cellulose. Sagittal whole-body cryosections (40 μm thick) were prepared using a Leica CM3600 cryomacrotome and collected onto adhesive tape (8″×3″, 810 Scotch® Brand Magic™ Tape, 3M) then freeze-dried within the chamber of the cryomacrotome. Prior to analysis using the SSSP, tissue sections were stored in a desiccator at room temperature. Color images of the tissue sections were acquired using a HP Scanjet 4370 flat-bed scanner (Hewlett-Packard, Palo Alto, Calif., USA).

Sealing Surface Sampling Probe Analysis:

The inlet and outlet of the SSSP (“TLC-MS interface”, CAMAG, Muttenz, Switzerland) were coupled to an ACQUITY UltraPerformance Liquid Chromatography (UPLC) system (Waters Corporation, Manchester, UK) and to a 4000 QTRAP® mass spectrometer (MDS SCIEX, Concord, Ontario, Canada), respectively (see FIG. S1 in the Supplemental section). The gas pressure applied via a piston to the plunger of the SSSP interface was about 5 bar. The flow rate of the extraction solvent was 200 μL/min in all cases. For positive ion mode ESI, the emitter voltage was 5 kV and the turbo sprayer heater temperature was 300° C. For positive ion mode APCI, heated nebulizer probe temperature and needle current were 350° C. and 5 μA, respectively. Ionization methods, extraction solvents, and MS conditions used for detection of each compound are listed in Table I. Scheme 1 shows the compound structures and the monitored precursor and product ions.

TABLE 1
Analytes, ionization methods, extraction solvent, SRM transitions and
mass spectrometer parameters settings including declustering potential
(DP) and collision energy (CE).

| | extraction | ionization | Q1 | Q3 | DP | CE |
|---|---|---|---|---|---|---|
| analyte | solvent | method | (m/z) | (m/z) | (V) | (eV) |
| propranolol | 80/20/0.1 | ESI+ | 260.1 | 183.1 | 60 | 27 |
| hydroxypropranolol | (v/v/v) | | 452.1 | 276.1 | 60 | 35 |
| glucuronide | acetonitrile/ | | | | | |
| | water/ | | | | | |
| | formic acid | | | | | |
| sitamaquine | 100/0.1 (v/v) | APCI+ | 344.4 | 271.2 | 42 | 30 |
| sitamaquine-d10 | methanol/ | | 354.4 | 271.2 | 42 | 30 |
| | formic acid | | | | | |
| acetaminophen | methanol | ESI+ | 152.1 | 110.1 | 24 | 40 |
| acetaminophen-d4 | | | 156.1 | 114.1 | 24 | 40 |

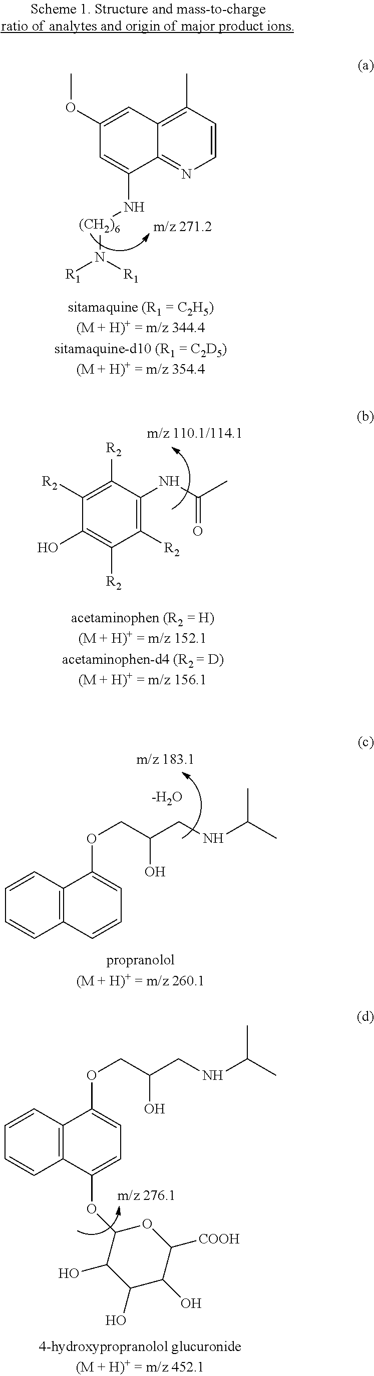

Operation of the SSSP:

The extraction solvent flow paths and the individual steps of the surface sampling process with the SSSP are illustrated in FIGS. 3A-3E and 4A-4E. The solvent inlet capillary to the SSSP was connected to the UPLC pump, while the outlet capillary was connected to the ion source (ESI or APCI) of the mass spectrometer. At the beginning of a surface sampling experiment, a sample was positioned under the SSSP plunger while the extraction solvent flowed from the UPLC through a loop into the ion source (as in FIGS. 3A and 4A). Next, the SSSP stainless steel plunger, with 4-mm-diameter disk shape cutting edge, was lowered to seal the sampling area by cutting into and compressing the sample substrate (paper or adhesive tape in this case) (as in FIGS. 3B and 4B). By switching the valve from standby to extraction mode, the solvent previously being pumped directly to the ion source was diverted to the surface, where it extracted the analyte, and any other soluble components, and carried them into the ion source (FIGS. 3C and 4C). After a suitable extraction time (typically 60 seconds), the valve was switched back to direct the extraction solvent from the UPLC through the loop into the ion source (FIGS. 3D and 4D). With the gas pressure of 5 bar applied via a piston to the plunger, and the surfaces and solvents being used, we found this seal would hold a backpressure up to about 13 bar without leaking significantly. Leaking was not an issue unless the fit on the downstream side of the sampling probe became clogged. In the final step of the surface sampling process, the plunger was raised up from the surface (FIGS. 3E and 4E) and then a cleaning station was positioned under the probe. Here, using push button control, the sampling face of the plunger was cleaned of accumulated particulate material using a stream of pressurized nitrogen. In the case of the tissue sections on tape, the probe actually cut out a section from the sample which stayed with the probe until removed by this gas jet cleaning. Because of the probe design, complete elimination of sample carryover required extraction at a blank spot (typically 60 seconds) on the sampling surface (or another blank surface). This washed the SSSP plunger face and tubing from the plunger to the valve and on into the ion source. Given 15 or 30 s to position the probe for analysis of a DBS or tissue sample spot, respectively, a 60 second extraction time and 60 s wash time to eliminate carryover, spot-to-spot analysis time was either 2.5 or 3 minutes.

Dried Blood Spot Analysis:

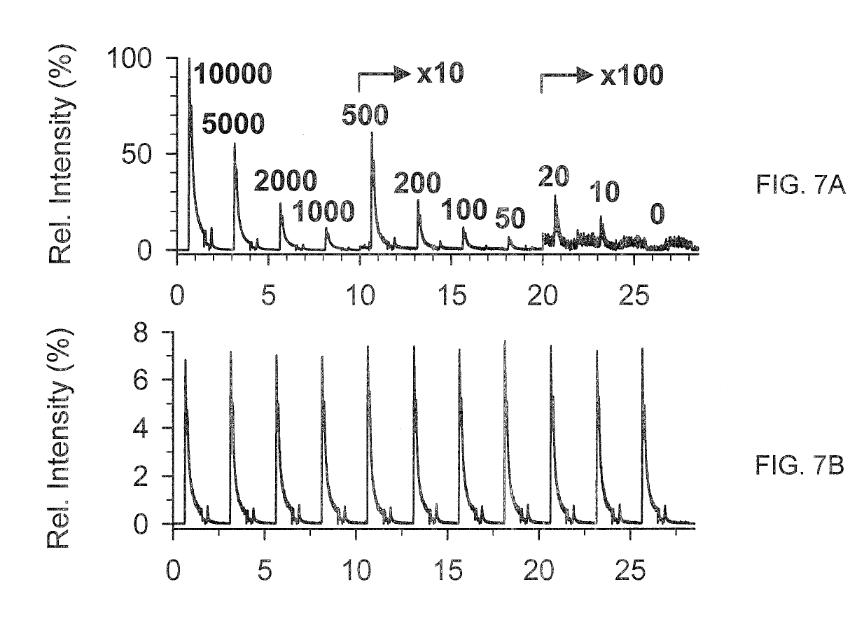

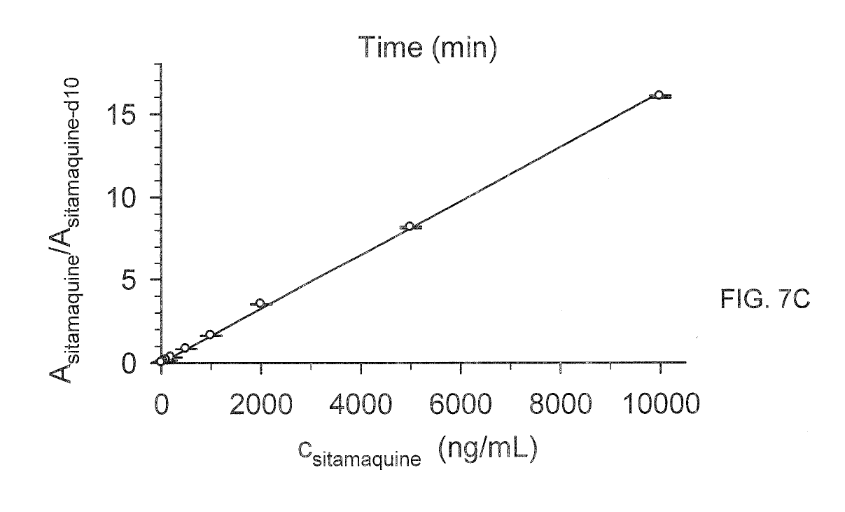

FIG. 7A shows the selected reaction monitoring (SRM) ion current chronogram for sitamaquine obtained from the analysis of each member of a calibration standard set of DBSs prepared from rat blood. Using positive ion mode ESI, each DBS sample was extracted for 60 seconds with a 100/0.1 (v/v) methanol/formic acid solvent at a flow rate of 200 μL/min. Inspection of these data showed the analyte extraction profiles to have an asymmetry similar to that which might be expected from a flow injection experiment. However, at least for the higher level standards, the signal for the drug was truncated before it completely reached background levels. This truncation was caused by switching the solvent flow path after the 60 second sampling period. To clear out this remaining material from the probe, and to clean the sampling head, the system was washed for 60 seconds between each standard/sample spot by sampling a blank spot on the DBS sample card. One also observed in these chronograms a small peak after almost all individual spot samples resulting from this washout.

The typical extraction profile and spot-to-spot signal reproducibility are more easily seen in FIG. 7B which presents the SRM signal for eleven replicates of the IS sitamaquine-d10 (570 ng/mL) collected simultaneously with the data shown in FIG. 7A. The coefficient of variation (CV) calculated from the integrated area of these IS peaks was 3.6%.

Using calibration standard data recorded before and after the analysis of QC samples, a calibration curve was constructed using the ratio of background corrected integrated SRM signal of sitamaquine and that of sitamaquine-d10 (A_{sitamaquine}/A_{sitamaquine}-d10) (over the 1 min sampling period) as a function of sitamaquine concentration (c_{sitamaquine}). The resulting calibration curve is plotted as the solid line in FIG. 7C. The averaged data (open circles) and associated error bars (CV) obtained from the separate analysis of the QC samples (n=6) are also plotted in FIG. 7C.

As summarized in Table 2 below, the precision of measurements (CV) was less than about 1% down to the 20 ng/mL QC samples, increasing to 3.4% for the 10 ng/mL samples. The back calculated concentrations for the QC standards generally showed an accuracy within 10% down to the 50 ng/mL level. Thus, this method provides the required accuracy and precision within internationally recognized acceptance criteria for assay validations down to the 50 ng/mL level.

TABLE 2
Nominal (c_{sitamaquine}) and calculated mean (c_{calc,} _{sitamaquine})
concentrations, precision (% CV, n = 6) and accuracy (% bias)
of sitamaquine quality control DBS samples based on a linear
fit of calibration standards.

| c_{sitamaquine} | c_{calc, sitamaquine} | precision | accuracy |
|---|---|---|---|
| (ng/mL) | (ng/mL) | (% CV) | (% bias) |
| 10000 | 9851.9 | 0.4 | −1.5 |
| 5000 | 5030.0 | 0.6 | 0.6 |
| 2000 | 2159.4 | 0.3 | 8.0 |
| 1000 | 1012.0 | 0.7 | 1.2 |
| 500 | 513.6 | 0.3 | 2.7 |
| 200 | 208.7 | 0.9 | 4.4 |
| 100 | 100.0 | 0.7 | 0.0 |
| 50 | 55.4 | 0.6 | 10.8 |
| 20 | 24.5 | 1.1 | 22.6 |
| 10 | 15.6 | 3.4 | 55.7 |

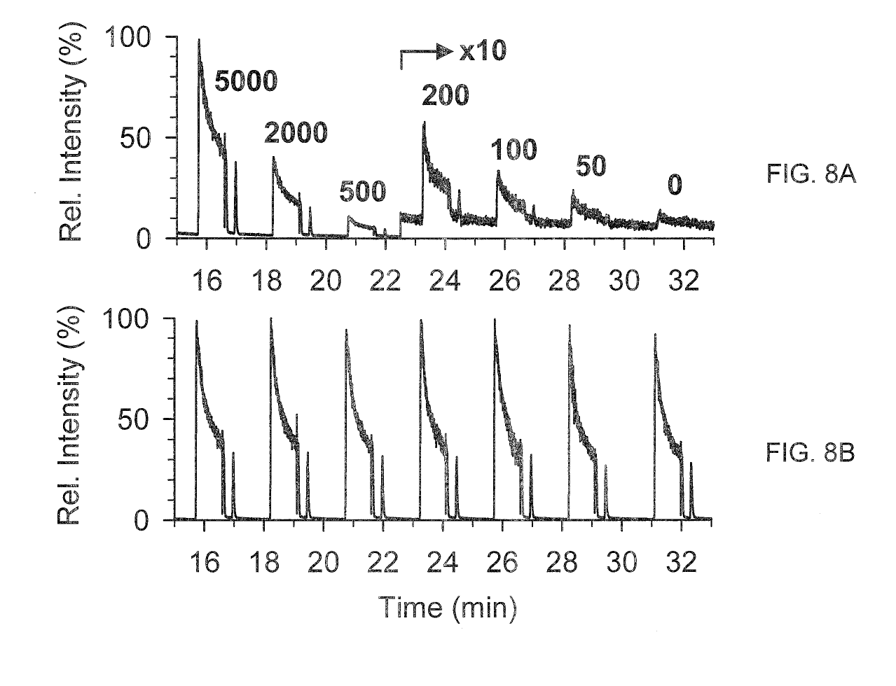

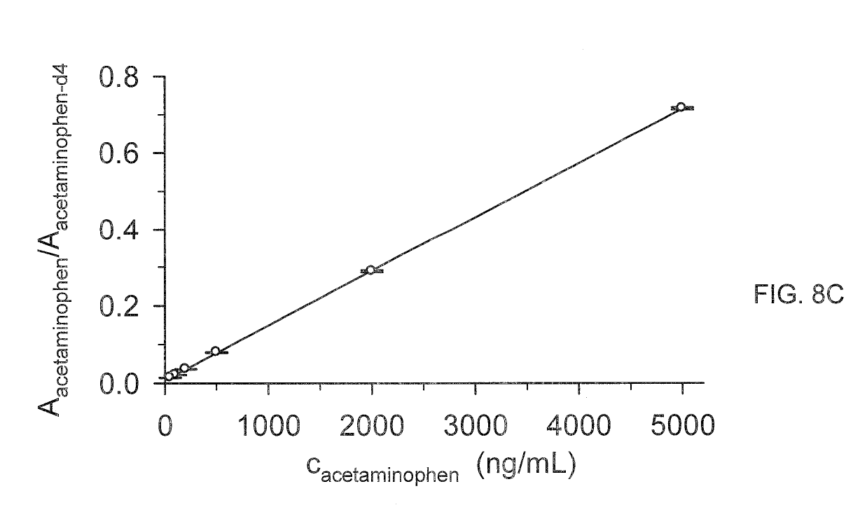

Analysis of DBS samples from human blood containing acetaminophen was also accomplished. We found that best results were obtained for analysis of this compound using 100% methanol as the extraction solvent (200 μL/min) and positive ion mode APCI. FIG. 8A shows the SRM ion current chronogram of acetaminophen for a calibration standard set. The analyte extraction profiles have the same basic appearance as those shown for sitamaquine in the experiments described above.

FIG. 8B presents the SRM signal for the IS acetaminophen-d4 (7000 ng/mL) collected simultaneously with that of acetaminophen. The CV calculated from the integrated area of these IS peaks was 6.0%.

A calibration curve constructed from the calibration standard data, recorded before and after the analysis of QC samples, using the ratio of background corrected integrated SRM signal of acetaminophen and that of its IS (Aacetaminophen/Aacetaminophen-d4) (over the 1 min sampling period) as a function of acetaminophen concentration (cacetaminophen) is presented as the solid line in FIG. 8C. The averaged data (open circles) and associated error bars (CV) obtained from the QC sample analysis (n=6) are also shown in FIG. 8C.

Statistical evaluation of the data summarized in Table 3 below shows that the precision of measurements (CV) was less than about 2% down to the 100 ng/mL QC samples, increasing to 4.1% for the 50 ng/mL samples. The back calculated concentrations for the QC standards showed an accuracy within 4% down to the lowest investigated 50 ng/mL level. These quantitation metrics results show that this method provides accuracy and precision values required by internationally recognized acceptance criteria for assay validations down to the 50 ng/mL acetaminophen level.

TABLE 3
Nominal (c_{acetaminophen}) and calculated mean (c_{calc, acetaminophen})
concentrations, precision (% CV, n = 6) and accuracy (% bias)
of acetaminophen quality control DBS samples based on a linear
fit of calibration standards.

| c_{acetaminophen} | c_{calc, acetaminophen} | precision | accuracy |
|---|---|---|---|
| (ng/mL) | (ng/mL) | (% CV) | (% bias) |
| 5000 | 4998.7 | 0.4 | 0.0 |
| 2000 | 1984.8 | 0.9 | −0.8 |
| 500 | 510.7 | 1.0 | 2.1 |
| 200 | 206.4 | 1.3 | 3.2 |
| 100 | 99.7 | 2.0 | −0.3 |
| 50 | 51.4 | 4.1 | 2.7 |

Thin Tissue Section Analysis:

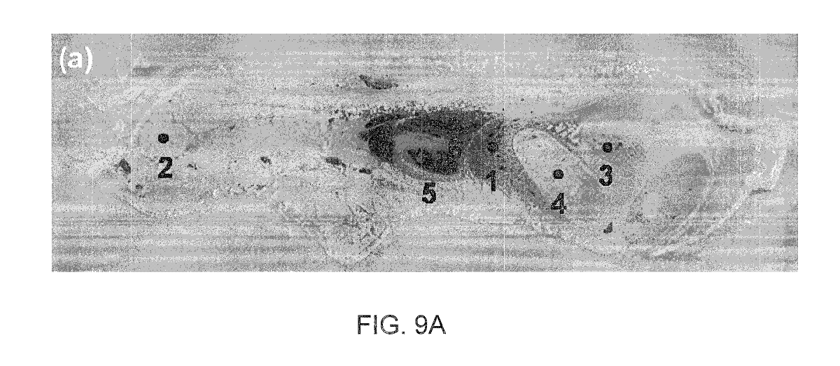

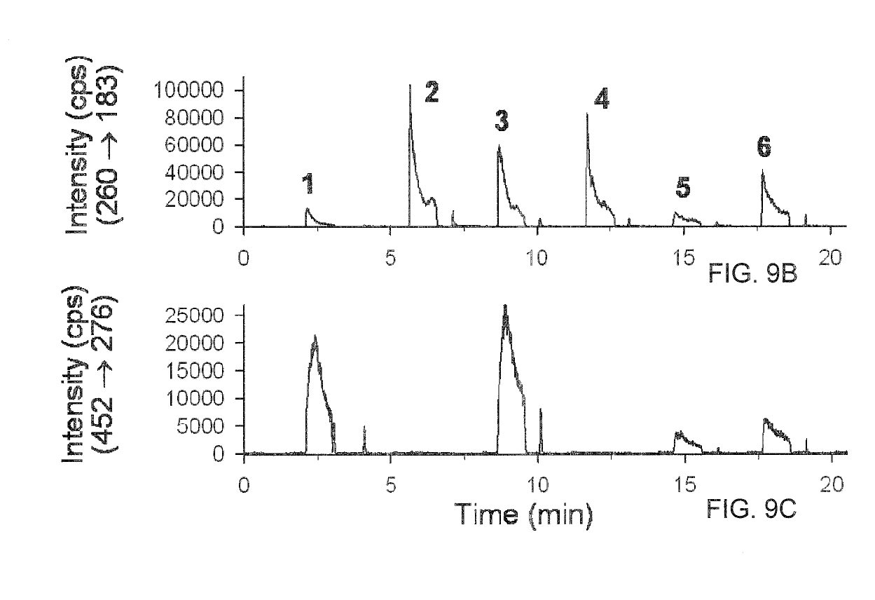

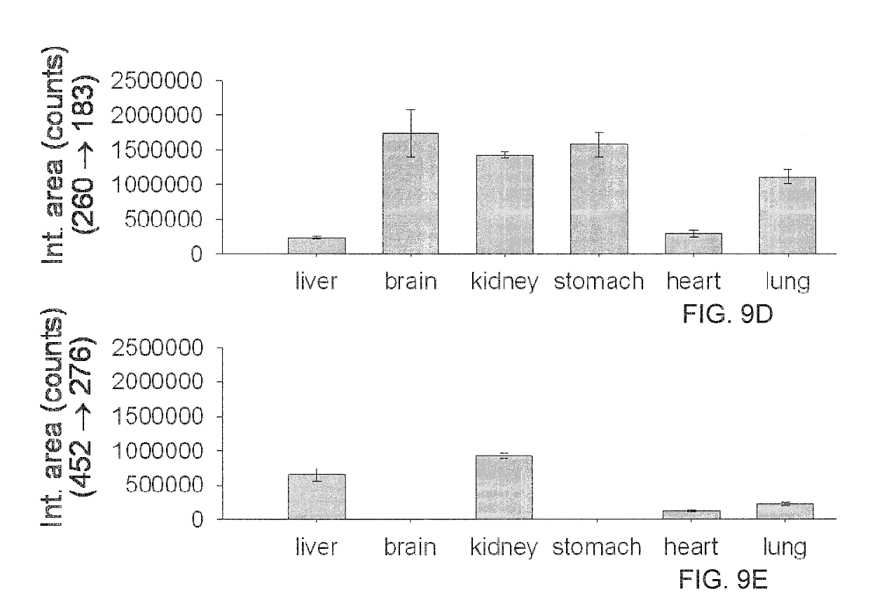

Spot sampling experiments were used to demonstrate the detection of targeted exogeneous compounds in mouse whole-body thin tissue sections. For this investigation, we used tissues sections from a mouse that had been administered 7.5 mg/kg propranolol intravenously via the tail vein and sacrificed 60 min later. This tissue section is shown in FIG. 9A. The tissue section was collected onto an adhesive tape substrate, which is a common format to collect whole-body tissue sections for WBA analyses. Using positive ion mode ESI and SRM detection, each sample spot was extracted for 1 min using a 80/20/0.1 (v/v/v) acetonitrile/water/formic acid at a flow rate of 200 μL/min. The areas sampled are annotated in the photograph of the tissue shown in FIG. 9A. Note that the probe actually cut out a 4-mm-diameter section from the tissue during the sampling process at each spot. These sample cutouts are not visible in the photograph because it was taken before the sampling process.

The SRM chronograms obtained for propranolol and its hydroxypropranolol glucuronide metabolite from the sequential sampling of the different organs are shown in FIGS. 9B and 9C, respectively. High propranolol signals were observed for brain, kidney and lung (spots 2, 3 and 6, respectively). In addition, significant level of the drug was detected in the stomach (spot 4). A much lower propranolol signal was recorded in the liver (spot 1) and lung (spot 5). As might be expected, signal levels for hydroxypropranolol glucuronide were highest in the liver and kidney (spots 1 and 3, respectively). Glucuronidation occurs mainly in the liver, making the drug more water-soluble to enhance subsequent elimination from the body by urination via the kidneys. Much lower hydroxypropranolol glucuronide signals were observed in the heart and lung (spots 5 and 6, respectively) and this metabolite was not detected in the brain (spot 2). These general observations are in line with the previously reported LMJ-SSP-MS/MS and WBA analyses of brain, liver, kidney and lung tissues of a mouse dosed following a protocol known in the art.

To show reproducibility of the sampling and signal levels from the various locations on the tissue, we examined another tissue section taken adjacent to and visually undistinguishable from the one examined above. SRM signals of the parent drug and that of the metabolite collected during the 1 min sampling period were integrated individually for all organs analyzed. FIGS. 9D and 9E show the averaged integrated areas and associated error bars (CV) calculated from the analysis results of the two tissues sections for propranolol and hydroxypropranolol glucuronide, respectively. The precision of measurements was typically around 10%, but always better than 20%. Though signal reproducibility is good, more work will be required to determine how well these signal levels reflect the actual quantities of the drugs and metabolites in each tissue. One might expect, for example, that the different composition of the various tissues might have a different matrix effect related to either extraction or ionization of these or any other targeted compounds.

In summary, a liquid extraction based SSSP has been applied for mass spectrometric analyses of targeted drugs in DBS samples on paper and targeted drugs and metabolites in thin tissue sections on an adhesive tape. Analysis of DBS samples employed both ESI and APCI. Quantitative evaluation of DBS samples containing a minimum of 50 ng/mL sitamaquine or acetaminophen resulted in accuracy and precision within internationally recognized acceptance criteria for assay validations. Further, whole-body thin tissue sections of mice intravenously dosed with pharmacologically relevant levels of the drug propranolol has been analyzed using the SSSP and ESI. Analysis of the thin tissue sections adhered to an adhesive tape substrate provided at the minimum a semi-quantitative abundance of propranolol and hydroxyproranolol glucuronide in the individual organs sampled. These relative abundance data were consistent with previous LMJ-SSP-MS/MS and WBA studies of tissues sections from mice subjected to a drug administration protocol known in the art. The successful sampling of tape-adhered tissue samples, which are generally employed in WBA analysis, demonstrated the possibility of a consecutive WBA and SSSP-MS analysis. This would facilitate molecular characterization of the total drug related material distributions provided by WBA.

While the invention has been described in terms of specific embodiments, it is evident in view of the foregoing description that numerous alternatives, modifications and variations will be apparent to those skilled in the art. Accordingly, the invention is intended to encompass all such alternatives, modifications and variations which fall within the scope and spirit of the invention and the following claims.

What is claimed is:

1. A system for generating a stream of eluate from a sample, said system comprising:
a sample assembly comprising a sample and a hydrophobic elastic layer contacting a back side surface of said sample; and
a sealing surface sampling probe configured to feed a liquid to a surface of a portion of said sample and to generate a stream of eluate, wherein said sealing surface sampling probe includes a knife edge configured to cut into a periphery of said portion of said sample to contact said hydrophobic elastic layer, wherein said portion of said sample is encapsulated by said hydrophobic elastic layer, said knife edge, and said liquid.

2. The system of claim 1, wherein said hydrophobic elastic layer consists essentially of at least one hydrophobic elastomer, at least one hydrophobic resin, or a combination thereof.

3. The system of claim 2, wherein said hydrophobic elastic layer is a hydrophobic elastic tape having a substantially uniform thickness.

4. The system of claim 3, wherein said hydrophobic elastic layer has a thickness from 300 micron to 6 mm.

5. The system of claim 1, wherein said sample assembly further includes a pad that provides mechanical support under external pressure of at least 200 psi, and said hydrophobic elastic layer is a hydrophobic coating located directly on a surface of said pad.

6. The system of claim 5, wherein said hydrophobic coating has a thickness from 3 microns to 300 microns.

7. The system of claim 1, wherein said sample assembly further comprises a substrate located on a back side of said hydrophobic elastic layer and providing mechanical support to said hydrophobic elastic layer.

8. The system of claim 1, wherein said portion of said sample is laterally enclosed by said knife edge and has a cross-sectional shape of a circle, an ellipse, a superellipse, or a polygonal shape.

9. The system of claim 1, wherein said portion of said sample is laterally enclosed by said knife edge and has an area from 2 mm² to 80 mm².

10. The system of claim 1, wherein said knife edge comprises a material selected from a metal or a metallic alloy.

11. The system of claim 1, wherein said sealing surface sampling probe includes at lease one inlet for letting in said liquid and an outlet for letting out said stream of eluate.

12. The system of claim 1, wherein said sample is a slice of a biological tissue.

13. The system of claim 12, wherein said biological tissue has a thickness from 4 microns to 400 microns.

14. The system of claim 1, wherein said liquid is a solvent that dissolves said portion of said sample.

15. A method of generating a stream of eluate from a sample, said method comprising:
forming a sample assembly comprising a sample and a hydrophobic elastic layer contacting a back side surface of said sample;
bringing a sealing surface sampling probe into contact with said sample assembly, wherein a knife edge cuts into a periphery of a portion of said sample to contact said hydrophobic elastic layer; and
generating a stream of eluate from said portion of said sample by feeding a liquid to said portion of said sample, wherein said portion of said sample is encapsulated by said hydrophobic elastic layer, said knife edge, and said liquid, and wherein said stream of eluate includes said liquid and materials of said portion of said sample.

16. The method of claim 15, wherein said hydrophobic elastic layer consists essentially of at least one hydrophobic elastomer, at least one hydrophobic resin, or a combination thereof.

17. The method of claim 16, wherein said hydrophobic elastic layer is a hydrophobic elastic tape having a substantially uniform thickness.

18. The method of claim 17, wherein said hydrophobic elastic layer has a thickness from 300 micron to 6 mm.

19. The method of claim 15, wherein said sample assembly further includes a pad that provides mechanical support under external pressure of at least 200 psi, and said hydrophobic elastic layer is a hydrophobic coating located directly on a surface of said pad.

20. The method of claim 19, wherein said hydrophobic coating has a thickness from 3 microns to 300 microns.

21. The method of claim 15, wherein said sample assembly further comprises a substrate located on a back side of said hydrophobic elastic layer and providing mechanical support to said hydrophobic elastic layer.

22. The method of claim 15, wherein said portion of said sample is laterally enclosed by said knife edge and has a cross-sectional shape of a circle, an ellipse, a superellipse, or a polygonal shape.

23. The method of claim 15, wherein said portion of said sample is laterally enclosed by said knife edge and has an area from 2 mm² to 80 mm².

24. The method of claim 15, wherein said knife edge comprises a material selected from a metal or a metallic alloy.

25. The method of claim 15, wherein said sealing surface sampling probe includes at lease one inlet for letting in said liquid and an outlet for letting out said stream of eluate.

26. The method of claim 15, wherein said sample is a slice of a biological tissue.

27. The method of claim 26, wherein said biological tissue has a thickness from 4 microns to 400 microns.

28. The system of claim 15, wherein said liquid is a solvent that dissolves said portion of said sample.

29. A system for generating a stream of eluate, said system comprising:
a sample assembly comprising a filter layer; and
a sealing surface sampling probe configured to feed a liquid to a surface of a portion of said filter layer and to generate a stream of eluate, wherein said scaling surface sampling probe includes a knife edge configured to cut into a periphery of said portion of said filter layer, wherein said portion of said filter layer is laterally confined by said knife edge.

30. The system of claim 29, wherein said sample assembly further includes a substrate contacting a back side surface of said filter layer.

31. The system of claim 30, wherein said substrate has a hydrophobic surface that contacts said back side surface of said filter layer.

32. The system of claim 29, wherein said portion of said filter layer is laterally enclosed by said knife edge and has a cross-sectional shape of a circle, an ellipse, a superellipse, or a polygonal shape.

33. The system of claim 29, wherein said knife edge comprises a material selected from a metal or a metallic alloy.

34. The system of claim 1, wherein said sample is a blood spot paper.

35. The system of claim 33, wherein said liquid is a solvent that dissolves blood.

36. A method of generating a stream of eluate, said method comprising:
forming a sample assembly comprising a filter layer;
bringing a sealing surface sampling probe into contact with said sample assembly, wherein a knife edge cuts into a periphery of a portion of said filter layer; and
generating a stream of eluate from said portion of said filter layer by feeding a liquid to said portion of said filter layer, wherein said portion of said filter layer is laterally confined by said knife edge, and wherein said stream of eluate includes said liquid and materials of said portion of said filter layer.

37. The method of claim 36, wherein said sample assembly further includes a substrate contacting a back side surface of said filter layer.

38. The method of claim 36, wherein said substrate has a hydrophobic surface that contacts said back side surface of said filter layer.

39. The method of claim 36, wherein said portion of said filter layer is laterally enclosed by said knife edge and has a cross-sectional shape of a circle, an ellipse, a superellipse, or a polygonal shape.

40. The method of claim 36, wherein said knife edge comprises a material selected from a metal or a metallic alloy.

41. The method of claim 36, wherein said sample is a blood spot paper.

42. The method of claim 41, wherein said liquid is a solvent that dissolves blood.

43. A system for analyzing a composition of an eluate, said system comprising:
a sample assembly comprising a filter layer or a combination of a sample and a hydrophobic elastic layer contacting a back side surface of said sample;
a sealing surface sampling probe configured to generate a stream of eluate from said sample assembly, wherein said sealing surface sampling probe includes a knife edge configured to laterally confine a portion of said sample or said filter layer, wherein said portion is laterally confined by said knife edge; and
an analytical instrument configured to receive and analyze said stream of eluate.

44. The system of claim 43, further comprising an ionization source configured to receive said stream of eluate from said sealing surface sampling probe and to provide an ionized material of said eluate to said analytical instrument.

45. A method of analyzing a composition of an eluate, said method comprising:
forming a sample assembly comprising a filter layer or a combination of a sample and a hydrophobic elastic layer contacting a back side surface of said sample;
bringing a sealing surface sampling probe into contact with said sample assembly, wherein a knife edge cuts into a periphery of a portion of said sample assembly;
generating a stream of eluate from said portion by feeding a liquid to said portion, wherein said portion is laterally confined by said knife edge, and wherein said stream of eluate includes said liquid and materials of said portion; and
analyzing said steam of eluate employing an analytical instrument.

46. The method of claim 45, further comprising:
generating an ionized material from said stream of eluate; and
providing said ionized material to said analytical instrument.
